(19) United States
(12) Patent Application Publication
(10) Pub. No.: US 2007/0213667 A1
(43) Pub. Date: Sep. 13, 2007

(54) Suction Irrigation Cleaner

(76) Inventor: Chad J. Prusmack, Denver, CO (US)

Correspondence Address: GLOBUS MEDICAL, INC.;ATTN: BRIAN MALM, VALLEY FORGE BUSINESS CENTER, 2560 GENERAL ARMISTEAD AVENUE, AUDUBON, PA, 19403, US

(21) Appl. No.: 11/683,372

(22) Filed: Mar. 7, 2007

Related U.S. Application Data
(60) Provisional application No. 60/779,981, filed on Mar. 7, 2006.

(51) Int. Cl. A61M 37/00

(52) U.S. Cl. 604/131

(57) ABSTRACT

A suction irrigation cleaner apparatus is disclosed.

### CROSS-REFERENCE TO RELATED APPLICATIONS

The present application claims priority to U.S. Provisional Application No. 60/779,981, filed Mar. 7, 2006, the entire contents of which are incorporated herein by reference.

### BACKGROUND OF THE INVENTION

In spine surgery, suction is important to evacuate blood and debris in the operative field to achieve certain goals. This is especially true in minimally invasive spine surgery where a very small access system or retractor is used. Here there is not much room or area to move the suction freely or use many tools simultaneously in the operative field. Commonly, tissue or clotted blood gets stuck in the suction itself, especially when a combination “suction-retractor” is used, which simultaneously achieves two goals:

1. Retracting a nerve root.

2. Continuous suction

When the suction becomes clotted or stuck, the surgeon must remove the suction from the operative field, which may be continuously bleeding, and have cleaned and irrigated most commonly by a surgical assistant. This is time consuming, frustrating, and sometimes even possibly dangerous.

### BRIEF DESCRIPTION OF THE DRAWINGS

The invention will be more readily understood with reference to the embodiments thereof illustrated in the attached figures, in which:

FIG. 1 is a perspective view of one embodiment of a suction irrigation cleaner apparatus according to the invention; and

FIG. 2 is a partial perspective view of another embodiment of a portion of the device of FIG. 1.

### DETAILED DESCRIPTION OF THE INVENTION

Some embodiments of the present device work with a valve system to clear the suction catheter without taking it from the operative field. One embodiment uses positive air or vapor pressure to do this and does not pose a danger to the tissue around it.

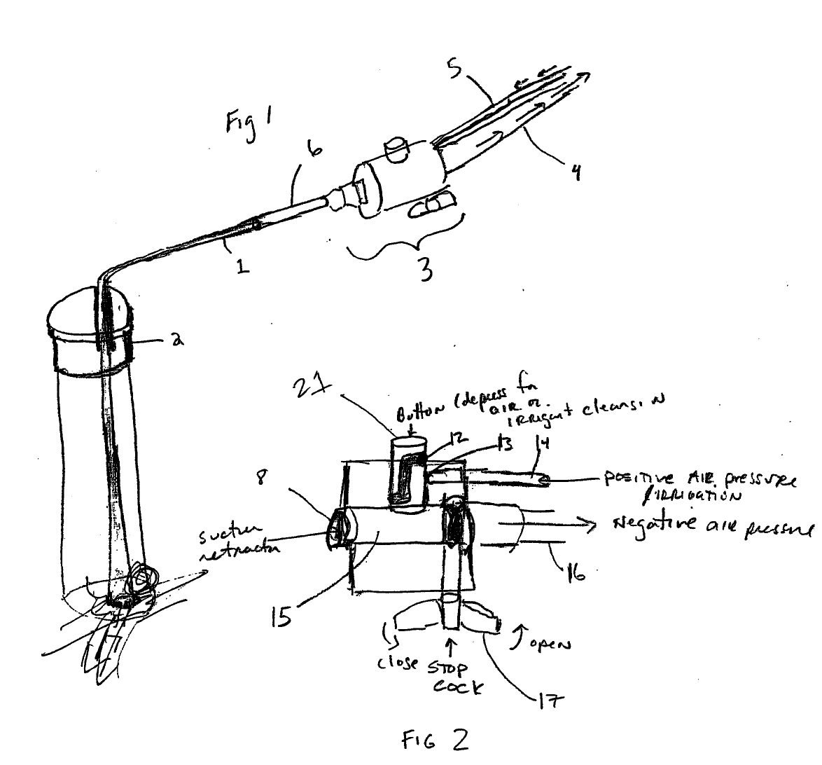

First referring to FIG. 1, one embodiment of a suction irrigation cleaner apparatus 3 according to the invention is shown. Cleaner 3 may be attached to a metal suction retractor 1 or even normal suction. In certain embodiments, suction retractor 1 may be made from a metal material and in minimally invasive spine surgery it may be attached to a minimally invasive access system or cannula 2. As shown in FIG. 1, one skilled in the art may appreciate that suction retractor 1 extends downward through the minimally invasive cannula 2 and serves its suctioning ability at distal end 7 adjacent the operative field. In one embodiment, catheter 6 is provided and may be made from a flexible rubber material which allows flexibility and mobility of the suction irrigation cleansing apparatus 3 independently from the rigid suction retractor for the surgeon's ease of use. In one embodiment, a vacuum suction source 4 may be attached to irrigation cleaner 3, and suction 4 may be attached to a wall suction port or other such port known to those skilled in the art and which typically available in an operating room.

In one variation, vacuum suction 4 may connect to a first or proximal end of cleaner 3. An additional positive pressure hose 5 may also connect to the proximal end of cleaner 3 and provide either positive air pressure or vapor pressure for either type of cleansing. In one variation, hose 5 enters a different port of the apparatus 3. One skilled in the art may appreciate that the overall function of cleaner 3 may be useful if distal end 7 were to become clogged, for example by tissue or clot, whereby one could use cleaner 3 to temporarily turn off the negative pressure or vacuum at 4, and then momentarily put positive pressure from hose 5 to clean out the tip of or distal end 7.

Referring to FIG. 2, another embodiment of a suction irrigation cleaner apparatus 3 is shown. In this embodiment, the orifice 8 is configured and dimensioned to attach to the rubber catheter 6, and that thereby attaches to the suction retractor as shown, for example, in FIG. 1.

In operation, under normal irrigation during the operation, the negative air pressure would go to a wall suction mechanism commonly used in operating rooms through an attachment port 16. In this regard, for normal suction, suction would come into the orifice 8, travel through 15, and exit through port 16 where the negative pressure originates.

In times when the retractor is “clogged”, the negative air pressure may be turned off locally by using a stopcock 17. In one embodiment, stopcock 17 may be a simple stopcock mechanism, which in a clockwise turn could open or close the opening 16, which is negative pressure. In a closed position, there would be no negative pressure in the chamber 15. Then once stopcock 17 is closed, in order to then introduce positive pressure, one could depress trigger 21, which may be a button. In depression of this button 21, it would cause a communication via the path 12. This would introduce positive pressure from the positive air pressure or water vapor area, which may be attached via a separate hose or port 14. So on depression of button 21, the positive air pressure would be introduced from port 14 through orifice 13 in continuity with the path 12, which would introduce it into the main chamber 15, and this would produce positive pressure into that chamber, which would be released into the rubber catheter 6 (shown in FIG. 1) then into 1, and then clear out the orifice at distal end 7. When one would let go or let the button 21 go back to its starting position, this would then disconnect the continuous path as seen in the mismatch of items 12 and 13 in FIG. 2. Then in order to resume normal negative suction air pressure, one could then move the stopcock 17 clockwise and resume normal suction. Alternatively, one mechanism, such as a button or valve, may control the functions of both the button 21 and valve stopcock 17.

Those skilled in the art may appreciate that one could achieve the surgical goals more quickly and safely by cleaning a suction, whether it be in an open surgery or in a minimally invasive spine surgery, and especially apply it to the combined suction retractor where you commonly get tissue and blood clotted at the orifice. This would be a safe way to quickly clean the suction retractor without the use of an assistant.

What is claimed is:

1. A suction irrigation cleaner apparatus, comprising:
a valve body extending from a proximal end to a distal end, wherein the proximal end is connectable to a first fluid source and second fluid source, and wherein the distal end is connectable to an extension extendable into a working cannula to an operative field,
the valve body defining a portion of a fluid pathway interconnecting the fluid sources to the operative field; and
a trigger mechanism connected to the valve body and moveable from a first position to a second position, wherein in a first position a first fluid path is defined and in the second position a second fluid path is defined.
